(19) United States
(12) Patent Application Publication
(10) Pub. No.: US 2024/0277619 A1
(43) Pub. Date: Aug. 22, 2024

(54) SPRAY DRYING OF API IN SUPERSATURATED SOLUTIONS WITH FORMIC ACID

(71) Applicant: Lonza Bend Inc., Bend, OR (US)

(72) Inventors: Michael Morgen, Bend, OR (US); Molly Adam, Bend, OR (US); John Baumann, Bend, OR (US); Nathan Bennette, Sisters, OR (US); Michael Grass, Bend, OR (US); Warren Miller, Bend, OR (US); Amanda Pluntze, Bend, OR (US); Daniel Regan, Bend, OR (US); David Vodak, Bend, OR (US)

(73) Assignee: Lonza Bend Inc., Bend, OR (US)

(21) Appl. No.: 18/568,197

(22) PCT Filed: Jun. 9, 2022
(86) PCT No.: PCT/EP2022/065703

§ 371 Date: Dec. 7, 2023

Related U.S. Application Data
(60) Provisional application No. 63/209,095, filed on Jun. 10, 2021.

(30) Foreign Application Priority Data
Jun. 15, 2021 (EP) 21179380.7

(51) Int. Cl. A61K 9/16; A61K 31/506; A61K 45/06

(52) U.S. Cl. CPC A61K 9/1682; A61K 9/1635; A61K 9/1652; A61K 31/506; A61K 45/06

(57) ABSTRACT

The invention discloses a method for preparation of spray dried solid dispersions (SDD) comprising an active pharmaceutical ingredient (API) and a dispersion polymer (DISPPOL), wherein the spray drying is done with a supersaturated solution of API in a solvent mixture comprising two solvents, this supersaturated solution further comprising DISPPOL.

The invention discloses a method for preparation of spray dried solid dispersions (SDD) comprising an active pharmaceutical ingredient (API) and a dispersion polymer (DISPPOL), wherein the spray drying is done with a supersaturated solution of API in a solvent mixture comprising two solvents, one of the two solvents is formic acid, this supersaturated solution further comprising DISPPOL.

### BACKGROUND OF THE INVENTION

Spray dried solid dispersions (SDD) comprising an active pharmaceutical ingredient (API) and a dispersion polymer (DISPPOL) are typically produced by dissolving the dispersion polymer and the API in a volatile solvent, such as methanol or acetone, or in a mixture of solvents, followed by spray drying. In cases where the API has limited solubility, e.g. <4 wt % at room temperature, in the spray drying solvent, an API suspension can be heated to a temperature either below or above the solvent's ambient pressure boiling point, this is known as “hot spray drying process”, resulting in a higher dissolved concentration of API. In some cases, even the higher temperatures do not give adequate API concentrations that are economical as a spray drying process, or cause other problems such as chemical degradation of the API, or the potential for incomplete API dissolution in the heat exchanger. Alternate, non-preferred volatile solvents can provide increased solubility of the API, but these solvents have other disadvantages that make them less desirable, e.g. high cost, toxicity, poor equipment compatibility, poor commercial availability, high disposal costs, challenges removing to sufficiently low levels.

Spray drying of suspensions is usually avoided since suspensions can lead to clogging of the nozzle of the spray dryer. Furthermore, when the intent of spray drying is the provision of an amorphous solid dispersion (ASD) of an API in a dispersion polymer, then this target is best achieved when both the API and the dispersion polymer are dissolved in the spray drying solvent so that both of them are not present in sold form in the spray drying mixture, thereby the desired intimate, homogenous and amorphous mixture of the ASD with the dispersion polymer is best obtained.

WO 2019/220282 A1 discloses in Example 1 spray drying of a solution of erlotinib and a dispersion polymer (PMMAMA or hydroxypropyl methylcellulose acetate succinate H grade) in methanol to provide a spray dried dispersion.

US 2020/0261449 A1 discloses spray drying of a mixture of nilotinib tartrate, HPMC-AS and tartaric acid in methanol.

US 2019/0083629 A1 discloses spray drying of a mixture of Vemurafenib or Itraconazolan as API, BSA as excipient, methanol and formic acid.

There was a need for a method for preparing spray dried solid dispersion of API and dispersion polymers, which allows for dissolving the APIs in easily processable spray drying solvents at modest temperature, i.e. a temperature below the ambient pressure boiling point, at sufficiently high concentrations to enable economical throughput of SDDs. The SDD should be stable over a longer period of time.

A Solvent-Shift route was found which involves dissolving API to a high concentration in formic acid, thereby providing a solution of the API in formic acid having a relatively high viscosity, then diluting this API solution with a preferred spray drying solvent having a relatively low viscosity, e.g. methanol, ethanol or acetone, and containing the desired dispersion polymer, at ratios that result in a metastable supersaturated solution of API in a solvent mixture containing the dispersion polymer and having a rather low viscosity and that can be efficiently spray dried. Metastable supersaturated solution of API in the solvent mixture means that the API is present in the solvent mixture in dissolved state, no solid API is present; in a supersaturated solution the API is present at a concentration above the thermodynamical equilibrium concentration.

Such a supersaturated solution cannot be prepared by simply adding API to the mixed solvent; it must be generated by mixing of two solvents containing dissolved API and dispersion polymer, respectively. An advantage is that by the dilution of the solution of the API in formic acid with the preferred spray drying solvent, the viscosity of the resulting metastable supersaturated solution can be chosen to be noticeably lower than the viscosity of a solution of the API in formic acid, that means the viscosity is rather in the range of the viscosity of a solution of the in the pure preferred spray drying solvent. Another advantage of the Solvent-Shift route is that it allows higher concentration of dissolved API in the spray drying solution relative to thermodynamic maximum solubility of the API in the spray drying solution, giving higher spray drying efficiency and higher throughput for the manufacturing of SDDs of APIs, especially for such APIs with a rather low solubility in typical spray drying solvents. The higher API and dispersion polymer concentrations in the spray drying solution may also allow for enhanced properties of the spray dried particles, e.g. larger particles giving advantages for dosage form manufacture or product recovery.

### Abbreviations and Definitions Used in this Specification

AA active agent. As used herein, the term “active agent” refers to a component that exerts a desired physiological effect on a mammal, including but not limited to humans. Synonymous terms include “active ingredient,” “active substance,” “active component,” “active pharmaceutical ingredient,” and “drug.”
Amorphous Substantially non-crystalline. Amorphous solids lack a definite crystalline structure and a sharp, well-defined melting point; instead, an amorphous solid melts gradually over a range of temperatures.
API active pharmaceutical ingredient API
Dispersion: A system in which particles are distributed in a continuous phase of a different composition. A solid dispersion is a system in which at least one solid component is distributed in another solid component.
DISPPOL dispersion polymer
HPMCAS Hydroxypropyl Methylcellulose Acetate Succinate, Hypromellose Acetate Succinate, CAS 71138-97-1
MIXSOL2DISPPOL a mixture of DISPPOL with a second solvent SOL2
pKa the pKa of a basic site of an organic Bronstedt base is the pH at which half of these basic sites are protonated. At a pH which is lower than this basic pKa more than half of these basic sites are protonated, that is ionized. This pKa of a basic site is also called basic pKa.
   In contrast thereof the pKa of an acidic site of an organic Bronstedt acid is the pH at which half of these acidic sites are deprotonated, that is ionized. At a pH which is higher than this acidic pKa more than half of these acidic sites are deprotonated. This pKa of an acidic site is also called acidic pKa.
   pKa values are available in the internet, they may also be calculated, for example by ADMET Predictor® software, Simulations Plus, Inc. (Nasdaq: SLP) or measured in the lab.
PPO polypropylenoxide
RT room temperature, for the purpose of the invention RT means temperatures from 20 to 25° C.
PXRD Powder X-Ray Diffraction
SDD Spray dried solid dispersions
SOL1 first solvent
SOL2 second solvent
solubility Solubilities stated herein in wt % mean weight of dissolved substance per weight of solvent;
   solubilities stated herein in mg/ml or in mg/g mean mg of dissolved substance per ml or per mg of solvent;
   any solubilities herein are determined at a given temperature, such as room temperature to 60° C., in particular at the given temperature which SUPSATSOL has when SUPSATSOL is fed into the spray dryer;
   if not stated explicitly otherwise.
Solubilize To make soluble or increase the solubility of.
Solution A homogeneous mixture composed of two or more substances. A solute (minor component) is dissolved in a solvent (major component). In contrast to a suspension, light passes through a solution without scattering from solute particles.
SOLUTION1 a solution of AA in a first solvent SOL1, optionally further comprising DISPPOL
SUPSATSOL supersaturated solution of an active agent AA in a solvent mixture SOLMIX further comprising DISPPOL. SUPSATSOL is a spray solution which is sprayed to provide a spray dried solid dispersion SDD comprising an active agent AA and a dispersion polymer DISPPOL. As used herein, the term “spray solution”, that is SUPSATSOL, refers to a fluid formed by dissolving an active agent and a dispersion polymer in a solvent and an amount of ammonia. In the case of the active agent, the term “dissolved” has the conventional meaning, indicating that the active agent has gone into solution when combined with the solvent and the amount of ammonia. In the case of dispersion polymers, the term “dissolved” can take a broader definition. For some dispersion polymers, the term dissolved can mean that the dispersion polymer has gone into solution and has dissolved in the conventional sense, or it can mean that the dispersion polymer is dispersed or highly swollen with the solvent such that it acts as if it were in solution, or it can mean that a portion of the dispersion polymer molecules are in solution and the remaining dispersion polymer molecules are dispersed or highly swollen with solvent. Any suitable technique may be used to determine if the active agent and dispersion polymer are dissolved. Examples include dynamic or static light scattering analysis, turbidity analysis, and visual observations.
SOLMIX the solvent mixture of SOL1 and SOL2 which is obtained when SOLUTION1 and MIXSOL2DISPPOL or SOL2 are mixed
Vitamin E TPGS Tocofersolan (INN), Vitamin E D-α-tocopheryl polyethylene glycol succinate
wt % any wt % value herein is based on the weight of the solution or mixture, if not explicitly stated otherwise

### SUMMARY OF THE INVENTION

Subject of the invention is a method SPRAYDRY for preparing a spray dried solid dispersion SDD comprising an active agent AA and a dispersion polymer DISPPOL;
the method SPRAYDRY comprising:
   providing a solution SOLUTION1 of AA in a first solvent SOL1,
   mixing SOLUTION1 with a second solvent SOL2 to provide a solution SUPSATSOL,
   spray drying of SUPSATSOL in a spray dryer;
      wherein
AA is a drug, medicament, pharmaceutical, therapeutic agent, nutraceutical or an active pharmaceutical ingredient;
SUPSATSOL comprises a solvent mixture SOLMIX and AA, with SOLMIX being the mixture of SOL1 and SOL2;
SUPSATSOL is a supersaturated solution of AA in SOLMIX; SUPSATSOL does not contain AA in solid form;
DISPPOL is contained in SOLUTION1, in SOL2 or in both prior to the mixing of SOLUTION1 with SOL2,
SOL1 comprises from 90 to 100 wt % of formic acid, with the wt % being based in the weight of SOL1;
AA is stable in SOL1, SOL2 and SOLMIX.

### DESCRIPTION OF THE DRAWINGS

FIG. 1: PXRD diffractogram showing the amorphous nature of the SDD, which represents a nilotinib dispersion in HPMCAS-MG.

FIG. 2: viscosities of a HPMCAS-M solution in various mixtures of formic acid and methanol.

### DETAILED DESCRIPTION OF THE INVENTION

Supersaturation in the sense of the invention means a concentration of AA in SOLMIX which is above the concentration of a saturated solution of AA in SOLMIX at a given temperature, in particular at the temperature of SUPSATSOL when SUPSATSOL is fed into the spray dryer; so the concentration of AA in SOLMIX is above the respective thermodynamical equilibrium concentration of AA in SOLMIX. SUPSATSOL is a metastable supersaturated solution of AA in SOLMIX. Metastable in the sense of the invention means that AA does not precipitate from SUPSATSOL between the preparation of SUPSATSOL and its spray drying. So AA is present in SUPSATSOL in a completely dissolved state. SUPSATSOL does not contain AA in solid form.

SUPSATSOL has only one liquid phase.

The amounts of AA, SOLMIX and DISPPOL may be chosen respectively. Supersaturation of AA in SUPSATSOL may also be expressed relative to the solubility of AA in SOLMIX; the concentration of AA dissolved in SUPSATSOL may be at least 1.1-fold, preferably at least 1.25-fold, more preferably at least 1.5-fold, even more preferably at least 1.75-fold, especially at least 2-fold, more especially at least 2.5-fold, even more especially at least 3-fold, in particular at least 4-fold, more in particular at least 5-fold, even more in particular cases even at least 10-fold, of the concentration of AA in a saturated solution of AA in SOLMIX, that is of the solubility of AA in SOLMIX, at a given temperature, in particular at the temperature of SUPSATSOL when SUPSATSOL is fed into the spray dryer.

Possible amounts of AA in SUPSATSOL may be from 0.5 wt % to 10 wt %, preferably from 1 wt % to 7.5 wt %, more preferably from 1 wt % to 5 wt %, with the wt % being based on the weight of SUPSATSOL.

When DISPPOL is comprised in SOL2 prior to the mixing of SOLUTION1 with SOL2 then this mixture of DISPPOL with SOL2 is called MIXSOL2DISPPOL herein.

Therefore,
in one embodiment, SUPSATSOL is prepared by mixing SOLUTION1, which is a solution of AA in SOL1, with MIXSOL2DISPPOL;
in another embodiment, SUPSATSOL is prepared by mixing SOLUTION1, which is a solution of AA in SOL1 comprising DISPPOL, with SOL2;
in yet another embodiment, SUPSATSOL is prepared by mixing SOLUTION1, which is a solution of AA in SOL1 comprising DISPPOL, with MIXSOL2DISPPOL;
preferably, SUPSATSOL is prepared by mixing SOLUTION1, which is a solution of AA in SOL1, with MIXSOL2DISPPOL.

The mixing of SOLUTION1 with MIXSOL2DISPPOL or with SOL2 to prepare SUPSATSOL may be done in any way that is known to the skilled person for the mixing of liquids, such as continuous mixing, for example by with an in-line mixer, such as a T shaped mixer, or by batch wise mixing, for example in a vessel.

In case of continuous mixing, the mixing and the spray drying of SUPSATSOL may be done continuously and consecutively, that is without any isolation or retainment of SUPSATSOL between the mixing and the spray drying. Thereby the time between the mixing and the spray drying of SUPSATSOL may be short, this time may be as short as a few milliseconds to seconds; this may be advantageous in case that the metastability of SUPSATSOL is only of short duration.

The mixing of SOLUTION1 with MIXSOL2DISPPOL or with SOL2 to prepare SUPSATSOL may be done
with SOLUTION1 having a temperature of from 4° C. to the boiling point of SOLUTION1 at ambient pressure, preferably from 4° C. to a temperature below the boiling point of SOLUTION1 at ambient pressure, in particular a given temperature which SUPSATSOL has when SUPSATSOL is fed into the spray dryer, and
with MIXSOL2DISPPOL or SOL2 having a temperature of from 4° C. to the boiling point of MIXSOL2DISPPOL at ambient pressure, preferably from 4° ° C. to a temperature below the boiling point of MIXSOL2DISPPOL at ambient pressure, in particular a given temperature which SUPSATSOL has when SUPSATSOL is fed into the spray dryer;
   preferably
with SOLUTION1 having a temperature of from 4 to 60° C., preferably from room temperature to 60° C., in particular a given temperature which SUPSATSOL has when SUPSATSOL is fed into the spray dryer, and
with MIXSOL2DISPPOL or SOL2 having a temperature of from 4 to 60° C., preferably from room temperature to 60° C., in particular a given temperature which SUPSATSOL has when SUPSATSOL is fed into the spray dryer.

SOLMIX is the solvent mixture of SOL1 and SOL2 which is obtained when SOLUTION1 is mixed with MIXSOL2DISPPOL or SOL2.

In an embodiment, SUPSATSOL consists of AA, DISPPOL, SOL1 and SOL2, with AA, DISPPOL, SOL1 and SOL2 as defined herein, also with their embodiments.

SDD is a spray dried solid dispersion of AA in DISPPOL. AA and DISPPOL are preferably homogeneously mixed in SDD.

In a solid dispersion of AA in DISPPOL, AA may be homogeneously and preferably also molecularly dispersed in DISPPOL. AA and DISPPOL may form a solid solution in SDD.

AA is amorphous or substantially amorphous in SDD; substantially means that at least 80 wt %, preferably at least 90 wt %, more preferably at least 95 wt %, even more preferably at least 98 wt %, especially at least 99% wt %, of AA is amorphous; the wt % being based on the total weight of AA in SDD. SDD therefore may be an amorphous SDD. The amorphous nature of AA may be evidenced by a lack of sharp Bragg diffraction peaks in the x-ray pattern when SDD is analyzed by a powder X-Ray Diffraction (PXRD). Possible parameters and settings for a x-ray diffractometer are equipment with a Cu-Kalpha source, setting in modified parallel beam geometry between 3 and 40° 2Theta and a scan rate of 2°/min with a 0.0° step size. Another evidence for the amorphous nature of AA in the SDD may be a single glass transition temperature (Tg). A single Tg is also evidence of a homogeneous mixture of amorphous AA and polymer. Samples as such without any further sample preparation may be used for the determination of the Tg, the determination may run for example in modulated mode at a scan rate of 2.5° C./min, modulation of +1.5° C./min, and a scan range from 0 to 180° C. Amorphous nature of AA shows a Tg which is equal to the Tg of neat DISPPOL or which is between the Tg of the polymer and the Tg of the AA. The Tg of the SDD is often similar to the weighted average of the Tg of AA and the Tg of DISPPOL. SDD is amorphous or substantially amorphous, SDD can also be called ASD.

The concentration of DISPPOL in SUPSATSOL may be above or below, preferably below the saturation concentration of DISPPOL in SOLMIX at a given temperature, in particular at the temperature of SUPSATSOL when SUPSATSOL is fed into the spray dryer.

In one embodiment, DISPPOL is present in SUPSATSOL in a dissolved state, the amounts of DISPPOL and SOLMIX are chosen respectively.

Amounts of DISPPOL in SUPSATSOL may be from 0.5 wt % to 25 wt %, preferably from 1 wt % to 20 wt %, more preferably from 2.5 wt % to 15 wt %, even more preferably from 5 wt % to 10 wt %, with the wt % being based on the weight of SUPSATSOL.

Amounts of DISPPOL and of AA in SUPSATSOL are chosen such that a predefined amount of DISPPOL and of AA in the SDD are provided.

The SDD may comprise from 1 to 99 wt %, preferably from 10 to 95 wt %, more preferably from 10 to 80 wt %, even more preferably from 20 to 60 wt %, of AA, the wt % being based on the weight of the SDD.

The SDD may comprise from 1 to 99 wt %, preferably from 20 to 90 wt %, more preferably from 40 to 80 wt %, of DISPPOL, the wt % being based on the weight of the SDD.

Preferably, the combined content of AA and DISPPOL in SDD is from 65 to 100 wt %, more preferably from 67.5 to 100 wt %, even more preferably from 80 to 100 wt %; especially from 90 to 100 wt %; more especially from 95 to 100 wt %;
the wt % being based on the weight of the SDD;
in one embodiment, the SDD consists of AA and DISPPOL.

Relative amounts of AA to DISPPOL in SDD may be from 50:1 to 1:50, preferably from 25:1 to 1:25, more preferably from 10:1 to 1:10 (w/w).

AA may be any biologically active compound. The biologically active compound may be desired to be administered to a patient in need of the active agent.

AA may be a drug, medicament, pharmaceutical, therapeutic agent, nutraceutical agrochemical, fertilizer, pesticide, herbicide, nutrient, or an active pharmaceutical ingredient (API); preferably an API.

AA may be a “small molecule,” generally having a molecular weight of 2000 Daltons or less.

AA may be a Bronstedt base with a basic pKA of at least 3 or greater, preferably of 4 or greater, more preferably of 5 or greater. Preferably, when AA is a Bronstedt base, then AA in its free base form is combined with the formic acid. AA may be present in SUPSATSOL in its free base form or in its protonated form. When AA is combined in its free base form with the formic acid in its free base form, then AA is obtained again in its free base form after the spray drying, so SPRAYDRY recovers and provides AA in its free base form, so essentially all of AA is obtained and is present in the SDD in its free base form.

AA may be nilotinib.

AA may be one or more active agents; SDD may contain one or more AA.

DISPPOL may comprise one or more dispersion polymers, preferably 1, 2, 3 or 4, more preferably 1, 2 or 3, even more preferably 1 or 2 dispersion polymers.

DISPPOL may be a pharmaceutically acceptable dispersion polymer.

Suitable DISPPOL include, but are not limited to, hydroxypropyl methylcellulose acetate succinate (HPMCAS), hydroxypropyl methylcellulose phthalate (HPMCP), hydroxypropyl methyl cellulose (HPMC), hydroxypropyl cellulose (HPC), cellulose acetate phthalate (CAP), carboxymethyl ethyl cellulose (CMEC), polyvinylpyrrolidone (PVP), poly(vinylpyrrolidone-co-vinyl acetate) (PVP-VA), poly(methacrylic acid-co-methyl methacrylate) (PMMAMA), poly(methacrylic acid-co-ethyl acrylate), [acetic acid ethenyl ester, polymer with 1-ethenylhexahydro-2H-azepin-2-one and .alpha.-hydro-.omega.-hydroxypoly(oxy-1,2-ethanediyl), graft], or any combination thereof.

Suitable PMMAMA polymers include, but are not limited to, poly(methacrylic acid-co-methyl methacrylate) 1:1 (for example Eudragit® L100), and poly(methacrylic acid-co-methyl methacrylate) 1:2 (for example Eudragit® S100). Eudragit® are polymer products of Evonik Industries AG, 45128 Essen, Germany.

The poly(methacrylic acid-co-ethyl acrylate) may be poly(methacrylic acid-co-ethyl acrylate) 1:1.

[Acetic acid ethenyl ester, polymer with 1-ethenylhexahydro-2H-azepin-2-one and .alpha.-hydro-.omega.-hydroxypoly(oxy-1,2-ethanediyl), graft] is a polymethacrylate and is available as Soluplus® from BASF, 67056 Ludwigshafen, GERMANY.

In a preferred embodiment, the dispersion polymer is chosen from HPMCAS, HPMC, PVP-VA, PVP, polymethacrylates, HPMCP, CMEC, CAP.

In another preferred embodiment, the dispersion polymer is chosen from HPMCAS, HPMC, PVP-VA, PVP, polymethacrylates, HPMCP, CMEC, CAP, [Acetic acid ethenyl ester, and polymer with 1-ethenylhexahydro-2H-azepin-2-one and .alpha.-hydro-.omega.-hydroxypoly(oxy-1,2-ethanediyl), graft].

In another more preferred embodiment, the dispersion polymer is chosen from HPMCAS, PVP-VA, polymethacrylates, HPMCP, CMEC, CAP.

In another preferred embodiment, the dispersion polymer is chosen from HPMCAS, PVP-VA, polymethacrylates, HPMCP, CMEC, CAP, [Acetic acid ethenyl ester, and polymer with 1-ethenylhexahydro-2H-azepin-2-one and .alpha.-hydro-.omega.-hydroxypoly(oxy-1,2-ethanediyl), graft].

In one embodiment the dispersion polymer is PMMAMA, HPMCAS, HPMC, PVP-VA or PVP;

in another embodiment the dispersion polymer is HPMC;

in another embodiment the dispersion polymer is PMMAMA, HPMCAS, PVP-VA or PVP;

in another embodiment the dispersion polymer is PMMAMA, HPMCAS or PVP-VA;

in another embodiment, DISPPOL is HPMCAS or PVP-VA;

in another embodiment the dispersion polymer is PVP-VA;

in another embodiment the dispersion polymer is PVP;

in another embodiment the dispersion polymer is HPMCAS.

Preferred embodiments of HPMCAS are
HPMCAS with an acetyl content from 5 to 9 wt % and a succinoyl content from 14 to 18 wt %,
HPMCAS with an acetyl content from 7 to 11 wt % and a succinoyl content from 10 to 14 wt %, or
HPMCAS with an acetyl content from 10 to 14 wt % and a succinoyl content from 4 to 8 wt %;
   more preferably
HPMCAS with an acetyl content from 5 to 7 wt % and a succinoyl content from 14 to 16 wt %,
HPMCAS with an acetyl content from 7 to 9 wt % and a succinoyl content from 10 to 12 wt %, or
HPMCAS with an acetyl content from 11 to 13 wt % and a succinoyl content from 5 to 7 wt %;
   with the wt % being based on the weight of HPMCAS.

SOL1 has only one liquid phase.

SOL1 may comprise from 90 to 100 wt %, preferably from 95 to 100 wt %, more preferably from 97.5 to 100 wt %, even more preferably from 98 to 100 wt %, especially from 99 to 100 wt %, of formic acid, with the wt % being based in the weight of SOL1.

Preferably, SOL1 consists of formic acid. The term “consists of formic acid” in the meaning of the invention comprises commercially available formic acid in the common technical grade, such as formic acid with a purity of 98%.

When SOL1 comprises less than 100 wt % of formic acid, then SOL1 may comprise besides formic acid further solvents such as water, methanol, ethanol, 1-propanol, 2-propanol, acetone, 2-butanone, THF, methyl acetate, ethyl acetate, dichloromethane, 1,3-dioxolane, or mixtures thereof;

preferably water, methanol, acetone, or mixtures thereof;
more preferably water.

SOLUTION1 is prepared by dissolving AA in SOL1 and optionally adding any DISPPOL.

SOLUTION1 may be prepared by dissolving AA in SOL1, and optionally adding any DISPPOL, at a temperature from 4° ° C. to the boiling point of SOL1 at ambient pressure, preferably from 4° C. to a temperature below the boiling point of SOL1 at ambient pressure, more preferably at a temperature from room temperature to 60° ° C., in particular at a given temperature which SUPSATSOL has when SUPSATSOL is fed into the spray dryer.

AA in SOLUTION1 is in a dissolved state in SOLUTION1, the amounts of AA and of SOL1 are chosen respectively.

The concentration of AA in SOLUTION1 is below the saturation concentration of AA in SOL1 at a given temperature, in particular at the temperature of SOLUTION1 when SOLUTION1 is mixed with MIXSOL2DISPPOL or with SOL2 respectively to provide SUPSATSOL.

In one embodiment, DISPPOL is present in SOLUTION1 in a dissolved state, the amount of DISPPOL is chosen respectively.

The concentration of DISPPOL in SOLUTION1 is preferably below the saturation concentration of DISPPOL in SOL1 at a given temperature, in particular at the temperature of SOLUTION1 when SOLUTION1 is mixed with MIXSOL2DISPPOL or with SOL2 respectively to provide SUPSATSOL.

Typical solubility of AA in SOL1 may be at least 1 wt %, preferably at least 2 wt %, more preferably at least 5 wt %, even more preferably at least 10 wt %, especially at least 20 wt %, of AA; with the wt % being based on the weight of SOLUTION1; the solubility of AA in SOL1 being preferably at given temperature a temperature of from 4° C. to the boiling point of SOL1 at ambient pressure, preferably from 4° C. to a temperature below the boiling point of SOL1 at ambient pressure, more preferably from room temperature to 60° C., in particular at a given temperature which SUPSATSOL has when SUPSATSOL is fed into the spray dryer. SOL1 may be chosen respectively.

Lower limit of the amount of AA in SOLUTION1 may be at least 0.5 wt %, preferably at least 1 wt %, more preferably at least 2.5 wt %, even more preferably at least 5 wt %, especially at least 7.5 wt %, more especially at least 10 wt %, even more especially at least 20 wt %, in particular at least 30 wt %, with the wt % being based on the weight of SOLUTION1.

The amount of AA in SOLUTION1 may be up to 50 wt %, preferably up to 40 wt %, even more preferably up to 35 wt %, with the wt % being based on the weight of SOLUTION1.

Any of the lower limit of the amount of AA in SOLUTION1 may be combined with any of the upper limit of the amount of AA in SOLUTION1.

For example, amounts of AA in SOLUTION1 may be from 0.5 to 50 wt %, preferably from 0.5 to 40 wt %, more preferably from 0.5 to 35 wt %, with the wt % being based on the weight of SOLUTION1.

SOL2 has only one liquid phase.

SOL2 is a solvent commonly used for spray drying.

SOL2 may comprise methanol, ethanol, 1-propanol, 2-propanol, acetone, 2-butanone, THF, methyl acetate, ethyl acetate, dichloromethane, 1,3-dioxolane, or mixtures thereof.

SOL2 may comprise water chosen in such an amount that SOL2 remains having only one liquid phase. The solubilities of water in the possible non-aqueous solvents of SOL2 are known. Depending on the possible non-aqueous solvent SOL2, SOL2 may comprise 30 wt % or less, preferably 27.5 wt % or less, more preferably 25 wt % or less, even more preferably 22.5 wt % or less, especially 20 wt % or less, more especially 15 wt % or less, even more especially 10 wt % or less, in particular 5 wt % or less, of water, the wt % being based in the weight of SOL2;
in an embodiment, SOL2 consists of methanol, ethanol, 1-propanol, 2-propanol, acetone, 2-butanone, THF, methyl acetate, ethyl acetate, dichloromethane, 1,3-dioxolane, water, or mixtures thereof;
preferably SOL2 comprises methanol, ethanol, acetone, or mixtures thereof, optionally also water, preferably with SOL2 comprising from 0 to 25 wt % water, the wt % being based in the weight of SOL2;
more preferably SOL2 consists of methanol, acetone, water, or mixtures thereof, preferably with SOL2 comprising from 0 to 25 wt % water, the wt % being based in the weight of SOL2;
even more preferably SOL2 consists of methanol or of a mixture of methanol with water, preferably with SOL2 comprising from 0 to 25 wt % water, the wt % being based in the weight of SOL2.

When the dispersion polymer is HPMC, preferably SOL2 comprises water; with the amount of water and all its embodiments as stated herein, for example from 10 to 30 wt %, or from 15 to 30 wt %, or from 20 to 30 wt %; the wt % being based in the weight of SOL2.

Preferably, AA has a low solubility in SOL2, such as in methanol, ethanol, acetone, especially in methanol, e.g. a solubility of 40 mg/ml or less, or of less than 30 mg/ml, or of less than 20 mg/l, or of less than 15 mg/ml, or of less than 10 mg/ml, or of less than 7.5 mg/ml, or of less than 5 mg/ml, or of less than 4 mg/ml, or of less than 3 mg/ml, or of less than 2 mg/ml, or of less than 1 mg/ml, or of less than 0.5 mg/ml, or of less than 0.25 mg/ml, in SOL2 at a given temperature, such as room temperature to 60° C., in particular a given temperature which SUPSATSOL has when SUPSATSOL is fed into the spray dryer.

MIXSOL2DISPPOL is prepared by mixing DISPPOL with SOL2.

MIXSOL2DISPPOL may be prepared by mixing DISPPOL with SOL2 at a temperature from 4° C. to the boiling point of SOL2 at ambient pressure, preferably from 4° C. to a temperature below the boiling point of SOL2 at ambient pressure, more preferably at a temperature from room temperature to 60° C., in particular at a given temperature which SUPSATSOL has when SUPSATSOL is fed into the spray dryer.

In one embodiment, DISPPOL is present in MIXSOL2DISPPOL in a dissolved state, the amounts of DISPPOL is chosen respectively.

The concentration of DISPPOL in MIXSOL2DISPPOL is preferably below the saturation concentration of DISPPOL in SOL2 respectively at a given temperature, in particular at the temperature of MIXSOL2DISPPOL when MIXSOL2DISPPOL is mixed with SOLUTION1 to provide SUPSATSOL.

SOL2 may have a boiling point at ambient pressure of 115° C. or less.

Amounts of DISPPOL in MIXSOL2DISPPOL or in SOLUTION1 may be from 0.5 wt % to 20 wt %, preferably from 1 wt % to 20 wt %, more preferably from 2.5 wt % to 15 wt %, even more preferably from 5 wt % to 10 wt %, with the wt % being based on the weight of MIXSOL2DISPPOL or of SOLUTION1 respectively.

AA may have a solubility in SOL1 that is at least 5-fold, preferably at least 10-fold, more preferably at least 50-fold, even more preferably at least 100-fold higher than the solubility of AA in SOL2 at a given temperature, in particular at the temperature of SOLUTION1 when SOLUTION1 is mixed with MIXSOL2DISPPOL or with SOL2 to provide SUPSATSOL; SOL1 and SOL2 may be chosen respectively.

The ratio (w:w) of the amounts of SOL1: SOL2, when SUPSATSOL is prepared by mixing SOLUTION1 with MIXSOL2DISPPOL or with SOL2, may be from 1:1 to 1:20, preferably from 1:2 to 1:20, more preferably from 1:5 to 1:20, even more preferably from 1:8 to 1:20, especially from 1:8 to 1:16, more especially from 1:8 to 1:15, in one particular embodiment from 1:8 to 1:10, in another particular embodiment from 1:13 to 1:15.

The lower limit of the amount of SOL1 in SOLMIX may be 5 wt %, preferably 6 wt %, more preferably 7.5 wt %, the wt % being based on the weight of SOLMIX.

The upper limit of the amount of SOL1 in SOLMIX may be 50 wt %, preferably 33 wt %, more preferably 25 wt %, even more preferably 20 wt %, especially 15 wt %, more especially 10 wt %, the wt % being based on the weight of SOLMIX.

For ranges of the amount of SOL1 in SOLMIX any of the lower limits may be combined with any of the upper limits, for example the amount of SOL1 in SOLMIX may be from 5 to 50 wt %, preferably from 5 to 33 wt %, more preferably from 5 to 25 wt %, even more preferably from 5 to 20 wt %, especially from 5 to 15 wt %, more especially from 5 to 10 wt %, the wt % being based on the weight of SOLMIX.

SUPSATSOL may be fed into the spray dryer with a given temperature of SUPSATSOL up to the boiling point of SUPSATSOL at ambient pressure; preferably with a given temperature of from 4° C. to the boiling point of SUPSATSOL at ambient pressure, preferably from 4° C. to a temperature below the boiling point of SUPSATSOL at ambient pressure, more preferably from room temperature to 60° C. In the context of this invention the term “SUPSATSOL may be fed into the spray dryer with a temperature of SUPSATSOL” means that “SUPSATSOL is spray dried with a temperature of SUPSATSOL”.

The spray drying of SUPSATSOL in the spray dryer evaporates both SOL1 and SOL2.

The temperatures
for preparing SOLUTION1,
for preparing MIXSOL2DISPPOL,
for preparing SUPSATSOL, and
for feeding SUPSATSOL into the spray dryer
   may be same of different, they may be a given temperature from room temperature to 60° C.; preferably, the temperature
for preparing SOLUTION1 may be from room temperature to 60° C.,
for preparing MIXSOL2DISPPOL may be room temperature,
for preparing SUPSATSOL may be from room temperature to 60° C., and
for feeding SUPSATSOL into the spray dryer may be from room temperature to 60° C.

The spray drying may be done with an inlet temperature of from 60 to 165° C., preferably from 80 to 165° C., more preferably from 80 to 140° C.

The spray drying may be done with an outlet temperature equal to or less than the boiling point of the solvent in SOLMIX that has the highest boiling point, such as with an outlet temperature from 20° ° C. to a temperature of 10° ° C. below the boiling point of the solvent in SOLMIX that has the highest boiling point.

The spray drying may be done with any inert gas commonly used for spray drying, such as nitrogen.

In one embodiment, SUPSATSOL does not contain bovine serum albumin (BSA). SUPSATSOL may further comprise a surfactant SURF.

SURF may be mixed with SUPSATSOL, or SURF may be mixed with SOLUTION1, with MIXSOL2DISPPOL, with SOL1 or with SOL2 before the preparation of SUPSATSOL. SURF may be for example a fatty acid and alkyl sulfonate; docusate sodium (available from Mallinckrodt Spec. Chern., St. Louis, Mo.), and polyoxyethylene sorbitan fatty acid esters (Tween®, available from ICI Americas Inc, Wilmington, Del., Liposorb® P-20, available from Lipochem Inc, Patterson, N.J., and Capmul® POE-0, available from Abitec Corp., Janesville, Wis.), and natural surfactants such as sodium taurocholic acid, 1-palmitoyl-2-oleoyl-sn-glycero-3-phosphocholine, lecithin, other phospholipids and mono- and diglycerides, vitamin E TPGS, PEO, PEO-PPO-PEO triblock copolymers (known under the tradename pluronics), and PEO (PEO are also called PEG, polyethyleneglycols (PEG)).

The amount of SURF may be up to 10 wt %, the wt % being based on the weight of SDD.

SUPSATSOL may further comprises pharmaceutically acceptable excipients, such as fillers, disintegrating agents, pigments, binders, lubricants, flavorants, and so forth which can be used for customary purposes and in typical amounts known to the person skilled on the art.

The viscosity of SUPSATSOL may be at least 2 times, preferably at least 3 times, lower than the viscosity of a mixture of DISPPOL in SOL1 which has the same concentration of DISPPOL as the concentration of DISPPOL is in SUPSATSOL.

The viscosity of SUPSATSOL may be at least 2 times, preferably at least 3 times, lower than the viscosity of SOLUTION1.

After the spray drying of SUPSATSOL the SDD may be submitted to a second drying in order to reduce the amount of any residual SOL1 or SOL2 in SDD. Secondary drying may be done using a tray dryer or any agitated dryer known to the skilled person for drying solids.

Preferably, the final SDD may have a content of SOL1 of 5000 ppm or less, preferably of 500 ppm or less, more preferably of 100 ppm or less.

Preferably, the final SDD may have a content of SOL2 of 5000 ppm or less, preferably of 500 ppm or less, more preferably of 100 ppm or less.

Further subject of the invention is a spray dried solid dispersion SDD; wherein the SDD is obtainable by the method SPRAYDRY;

with SDD and SPRAYDRY as defined herein, also with all their embodiments.

### Examples

### Materials and Abbreviations

cP centipoise, centipoise is equal to the SI millipascal seconds (mPa·s)
formic acid 98%, EMD Millipore Corporation, an affiliate of Merck KGaA, Darmstadt, Germany
HPMCAS-MG HPMCAS in form of AQOAT® MG (also called AS-MG) was purchased from Shin-Etsu Chemical Co., Ltd. (Tokyo, Japan). The letter M specifies the grade and distinguish the contents of acetyl and succinoyl groups. Other grades are designated with the letters L (HPMCAS-L) and H (HPMCAS-H). The Letter G represents granular grade with a Mean Particle Size of 1 mm, a letter F instead of a G would represent micronized grade with a Mean Particle Size of 5 micrometer. Various contents and parameters of these grades are given in Table 3.

TABLE 3

| | | | Hydroxy- | Acetyl | Succinoyl | |
|---|---|---|---|---|---|---|
| | Viscosity | Methoxy | propoxy | content [wt %] | content [wt %] | Tg |
| | (mPa*s) | content | content | range/preferred | range/preferred | [° C.] |
| Grade | (a) | [wt %](c) | [wt %](c) | (c) | (c) | (b) |
| L | 2.4 to 3.6 | 20 to 24 | 5 to 9 | 5 to 9/6 | 14 to 18/15 | 122 |
| M | 2.4 to 3.6 | 21 to 25 | 5 to 9 | 7 to 11/8 | 10 to 14/11 | 122 |
| H | 2.4 to 3.6 | 22 to 26 | 6 to 10 | 10 to 14/12 | 4 to 8/6 | 122 |

(a) Viscosity of 2 w/w % solution of sodium hydroxide aqueous solution at 20° C.
(b) Tg of the HPMCAS was determined by DSC experiment under the following test condition:
Equipment: DSC Q2000 (TA Instruments. Japan)
Heating rate: 10° C./min
Referred to the second heating run
N₂ gas atmosphere
Sample size 3 mg
(c) the wt % based on the weight of the HPMCAS

NA not available
Nilotinib CAS 641571-10-0; MW 529.5 g/mol, Nilotinib, Free Base, >99%, was purchased from LC Laboratories, Woburn, MA 01801, USA. The term “nilotinib” refers to the free base form throughout the examples, if not explicitly stated otherwise.
   Nilotinib has two basic pKA: a basic pKA of 2.1 and a basic pKA of 5.4 (according to https://go.drugbank.com/drugs/DB04868 the strongest basic pKa is 5.92).
   At each of these basic pKA half of the respective basic site is protonated.

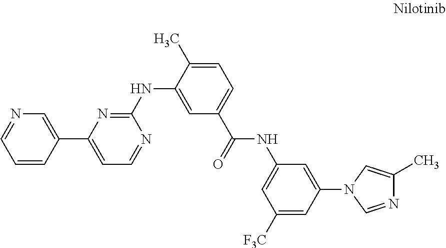 [SMILES: Cc1cn(-c2cc(NC(=O)c3ccc(C)c(Nc4nccc(-c5cccnc5)n4)c3)cc(C(F)(F)F)c2)cn1]

PVP-VA64 Kollidon® VA64, Vinylpyrrolidone-vinyl acetate copolymer, CAS 25086-89-9, PVP/VA Copolymer, BASF, Ludwigshafen, Germany

### Methods

### Viscosity of Solutions of Polymers in Solvents

Table 2 shows viscosities of a 7.4 wt % solution of HPMCAS-M in a mixture of formic acid and methanol. An amount of formic acid: MeOH of more than about 50:50 w:w shows a non linear increase in viscosity.

TABLE 2

| | viscosity of a |
|---|---|
| Formic acid: | solution of |
| MeOH | 7.4 wt % HPMCAS-M |
| w:w | [cP] at RT |
| 10:90 | 15.3 |
| 30:70 | 21.2 |
| 70:30 | 48.4 |
| 90:10 | 96.4 |

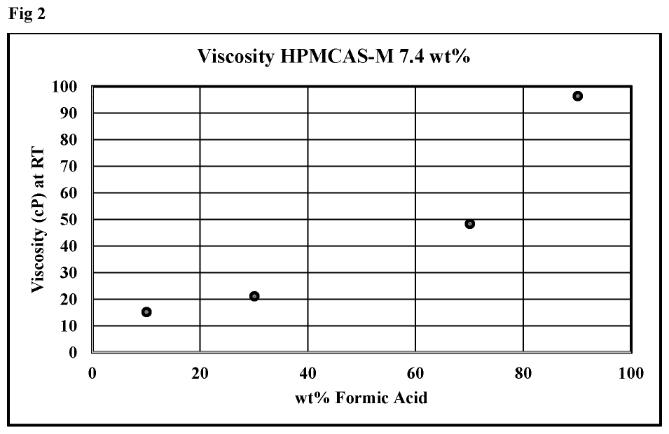

FIG. 2 shows data of Table 2 in graphical form, the viscosities of a HPMCAS-M solution in various mixtures of formic acid and methanol.

### Example 1: SDD Using Formic Acid for Solvent Shift with Methanol—25:75 Nilotinib: HPMCAS-MG

4.0 g of formic acid as SOL1 was weighed into flask 1 and 36.0 g of methanol as SOL2 was weighed into flask 2. 1.0055 g of nilotinib free base was added to flask 1 with formic acid and stirred with a magnetic stir bar (20)° ° C., resulting in a 20 wt % solution. 3.0040 g of HPMCAS-MG was added to flask 2 with methanol and stirred the same way as flask 1, resulting in a 7.7 wt % solution. Once both solutions were fully dissolved, the contents of flask 1 were slowly poured into flask 2 as it was being stirred providing a supersaturated solution SUPSATSOL of nilotinib in SOLMIX which was 9:1 (w:w) methanol:formic acid. This supersaturated solution had a nilotinib concentration of 2.28 wt % and a HPMCAS-MG concentration of 6.83 wt % in SOLMIX.

This supersaturated solution was stirred for 6 minutes before spray drying, it did not contain nilotinib in solid form, instead it contained the nilotinib in a completely dissolved state, and it had only one liquid phase.

This supersaturated solution with a temperature of 20° C. was spray dried using a custom built spray dryer. This supersaturated solution was pumped into a lab-scale 0.3 m diameter stainless steel spray drying chamber using a peristaltic pump to feed the solution to the nozzle at a flowrate of 15 g/min. A two-fluid nozzle ¼ J series with a 1650 liquid body and a 64 air cap made by Spraying Systems Company, Glendale Heights, IL 60187-7901, United States. Nitrogen gas as sheath gas was used to atomize the solution at a pressure of 20 psi. Heated nitrogen gas (115° C. inlet, 45 to 50° C. outlet, 500 g/min) was used to dry the particles. The resulting SDD was collected using a cyclone to separate the solids from the gas stream.

The collected SDD was placed in a vacuum tray dryer at 40° ° C. for secondary drying with 3.5 slpm (Standard-Liter per minute) nitrogen sweep for 24 h at a pressure of 0.2 atm.

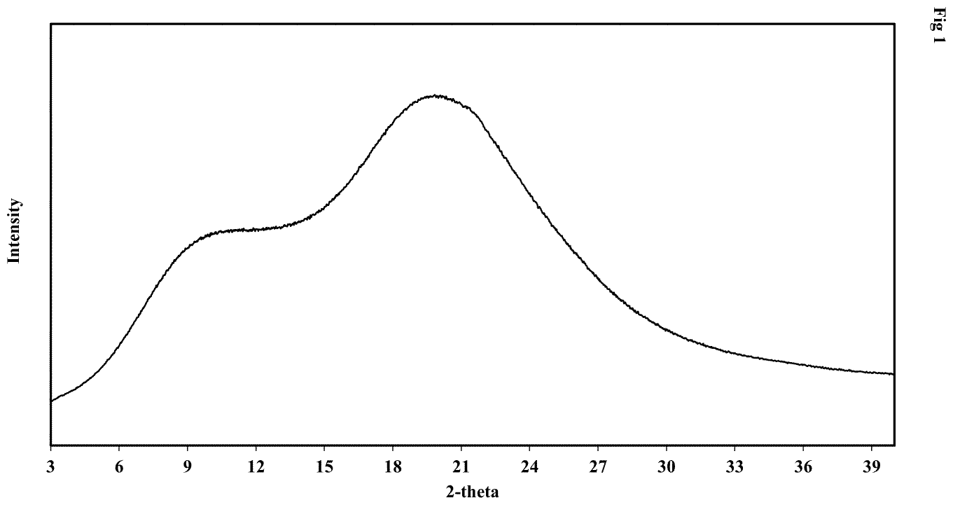

FIG. 1 shows the PXRD of the collected SDD, which represents a nilotinib dispersion in HPMCAS-MG, and it confirms the amorphous nature of the SDD.

### Comparative Example A

A mixture of the same composition as Example 1 was prepared not using the solvent shift method. In a 20 ml glass vial with magnetic stir bar, nilotinib free base, 0.100 g and HPMCAS-MG 0.301 g were combined. To this mixture was added 3.99 g of premixed 9:1 methanol:formic acid. The mixture was stirred for 24 h at 25° C. The sample did not dissolve, but remained heterogeneous with substantial solids present. The composition of the mixture (as wt %) was: 2.28% nilotinib, 6.83% HPMCAS and the remainder is solvent.

1. A method SPRAYDRY for preparing a spray dried solid dispersion comprising an active agent and a dispersion polymer, the method comprising:
providing a first solution of the active agent in a first solvent;
mixing the first solution with a second solvent to provide a second solution; and
spray drying the second solution in a spray dryer to form a spray dried solid dispersion,
wherein
the active agent is a drug, a medicament, a pharmaceutical, a therapeutic agent, a nutraceutical or an active pharmaceutical ingredient,
the second solution comprises a solvent mixture and the active agent, with the solvent mixture being a mixture of the first solvent and the second solvent;
the second solution is a supersaturated solution of the active agent in the solvent mixture;
the second solution does not contain the active agent in solid form;
the dispersion polymer is contained in the first solution, in the second solvent, or in both prior to the mixing of the first solution with the second solvent,
the first solvent comprises from 90 to 100 wt % of formic acid, with the wt % being based on a weight of the first solvent;
the active agent is stable in the first solvent, the second solvent, and the solvent mixture.

2. The method according to claim 1, wherein the dispersion polymer is a pharmaceutically acceptable dispersion polymer.

3. The method according to claim 1, wherein the dispersion polymer comprises hydroxypropyl methylcellulose acetate succinate (HPMCAS), hydroxypropyl methylcellulose phthalate, hydroxypropyl methyl cellulose, hydroxypropyl cellulose, cellulose acetate phthalate, carboxymethyl ethyl cellulose, polyvinylpyrrolidone, poly(vinylpyrrolidone-co-vinyl acetate) (PVP-VA), poly(methacrylic acid-co-methyl methacrylate), poly(methacrylic acid-co-ethyl acrylate), or any combination thereof.

4. The method according to claim 1, wherein the dispersion polymer is HPMCAS or PVP-VA.

5. The method according to claim 1, wherein the first solvent consists of formic acid.

6. The method according to claim 1, wherein the second solvent comprises methanol, ethanol, 1-propanol, 2-propanol, acetone, 2-butanone, tetrahydrofuran (THF), methyl acetate, ethyl acetate, dichloromethane, 1,3-dioxolane, or mixtures thereof.

7. The method according to claim 1, wherein the second solvent comprises methanol, ethanol, acetone, or any mixture thereof.

8. The method according to claim 1, wherein a ratio (w:w) of amounts of solvent 1: solvent 2, when the second solution is prepared by mixing the first solution with a mixture of the dispersion polymer and the second solvent or with the second solvent, is from 1:1 to 1:20.

9. The method according to claim 7, wherein the second solvent further comprises 0 wt % to 25 wt % water, based on a weight of the second solvent.

10. The method according to claim 1, wherein a concentration of the active agent dissolved in the second solution is at least 1.1-fold a concentration of the active agent in a saturated solution of the active agent in the solvent mixture at a given temperature.

11. The method according to claim 1, wherein a concentration of the active agent dissolved in the second solution is at least 2-fold a concentration of the active agent in a saturated solution of the active agent in the solvent mixture at a given temperature.

12. The method according to claim 1, wherein the second solution comprises from 0.5 wt % to 10 wt % of the active agent, based on a weight of the second solution.

13. The method according to claim 12, wherein the second solution comprises from 1 wt % to 7.5 wt % of the active agent.

14. The method according to claim 1, wherein a concentration of the dispersion polymer in the second solution is from 0.5 wt % to 25 wt %, based on a weight of the second solution.

15. The method according to claim 17, wherein a concentration of the dispersion polymer in the second solution is from 1 wt % to 20 wt %.

17. The method according to claim 1, wherein an amount of the first solvent in the solvent mixture is from 5 wt % to 50 wt %, based on a weight of the solvent mixture.

18. The method according to claim 1, wherein:
(i) a temperature for preparing the first solution is from room temperature to 60° C.;
(ii) a temperature for preparing a mixture of the dispersion polymer and the second solvent is from room temperature to 60° C.;
(iii) a temperature for preparing the second solution is from room temperature to 60° C.;
(iv) a temperature for feeding the second solution into the spray dryer is from room temperature to 60° C.; or
(v) any combination of two or more of (i)-(iv).

19. The method according to claim 1, wherein at least 80 wt % of the active agent is amorphous in the spray dried solid dispersion.

20. The method according to claim 1, wherein a combined content of the active agent and the dispersion polymer in the spray dried solid dispersion is from 65 wt % to 100 wt %, based on a weight of the spray dried solid dispersion.
